(19) United States
(12) Patent Application Publication
(10) Pub. No.: US 2021/0322592 A1
(43) Pub. Date: Oct. 21, 2021

(54) SANITIZING WORKSPACES USING ULTRAVIOLET LIGHT BUILT INTO A VIEWING DEVICE

(71) Applicant: Goldman Sachs & Co. LLC, New York, NY (US)

(72) Inventors: Michael Mattioli, Staten Island, NY (US); Frederick Biga, Jersey City, NJ (US)

(21) Appl. No.: 17/156,539

(22) Filed: Jan. 23, 2021

(51) Int. Cl. A61L 2/10; G09G 3/32

(52) U.S. Cl. CPC A61L 2/10; G09G 3/32; A61L 2202/11; G09G 2354/00; A61L 2202/25; G09G 2370/22; A61L 2202/14

(57) ABSTRACT

A four light emitting diode (LED) arrangement in a monitor (e.g., red, green, blue, and ultraviolet light emitting LEDs) enables a cleaning mode where all of the LEDs turn off except ones that produce UV light. In the cleaning mode, the monitor emits UV light to sanitize the workspace immediately around the display (e.g. desktop, keyboard, mouse, etc.). In larger conference room settings, the larger displays sanitize a part of the room in a cleaning mode because the larger display is angled and positioned to be seen from almost every angle of the room.

### TECHNICAL FIELD

This disclosure generally relates to display devices (e.g., viewing devices), and more particularly to sanitizing workspaces using a display device.

### BACKGROUND

A workspace including a display device may be shared by different users. It would be advantageous to sanitize the workspace in between use from different users for hygienic reasons. A common way of sanitizing a workspace is by manually cleaning the workspace with consumables such as disinfectant wipes or sprays. However, manually cleaning the workspace is inconvenient because it takes time and resources to schedule and implement the cleaning or time from users to sanitize the workspace, and to continually provide the sanitizing equipment.

### SUMMARY

This disclosure describes a mechanism for sanitizing a workspace using a display device. The display device may include a display panel and a controller. The display panel may include sets of light emitting diode (LED) units. Each set may include multiple visible light emitting LEDs and an ultraviolet (UV) light emitting LED. Visible light emitting LEDs may include LEDs that emit red, green, or blue light. The display panel may operate in a display mode (e.g., display image data with the visible light emitting LEDs) or a cleaning mode (e.g., sanitize a workspace using the UV light emitting LEDs). When the controller receives image data from a computing device, the controller causes the display panel to display the image data in a display mode. When the controller determines that no other image data has been received (e.g., that a connected computing device is not sending display data to the display device), the controller may retrieve one or more conditions for switching modes of operation to a cleaning mode. An example of a condition may be that the display device has been idle for a period of time (e.g., display device has not been used to display data) and/or no motion has been detected for a threshold period of time (e.g., indicating that no one is around and it would be safe to switch into a cleaning mode). When the controller determines that a condition for switching modes of operation to the cleaning mode has been met, the controller may cause the display panel to switch to the cleaning mode of operation, by turning off the visible light emitting LEDs and turning on the UV light emitting LED in each set of LED units to sanitize an area around the display device.

### BRIEF DESCRIPTION OF DRAWINGS

The disclosed embodiments have other advantages and features which will be more readily apparent from the detailed description, the appended claims, and the accompanying figures (or drawings). A brief introduction of the figures is below.

FIG. 1 illustrates one embodiment of a display device that is configured to sanitize an area around the display device.

FIG. 2 illustrates one embodiment of a controller of a display device.

FIG. 3 illustrates a data structure and example entries for a condition for switching mode of operations for a display panel, in accordance with some embodiments of this disclosure.

FIG. 4 illustrates one embodiment of a set of LED units of a display panel of a display device.

FIG. 5 is a block diagram illustrating components of an example machine able to read instructions from a machine-readable medium and execute them in a processor (or controller).

FIG. 6 illustrates one example of actions that a controller of a display device may perform to switch operation of a display panel from a display mode of operation to a cleaning mode of operation, in accordance with some embodiments of this disclosure.

### DETAILED DESCRIPTION

The figures and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

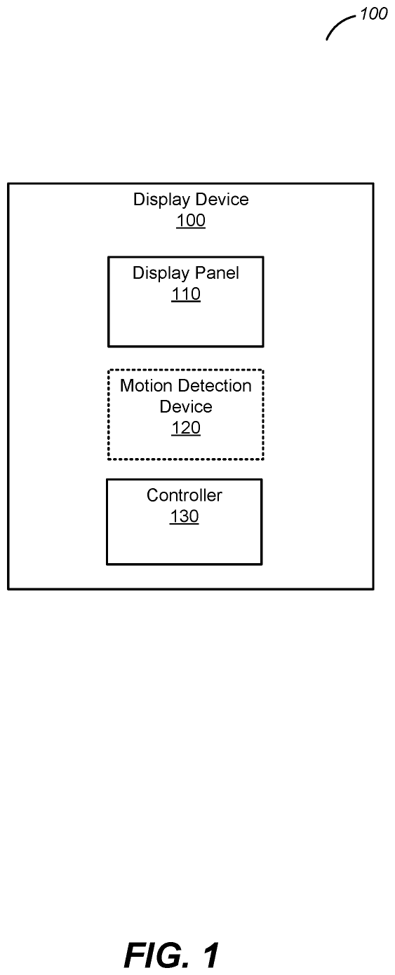

FIG. 1 illustrates one embodiment of a display device 100 that is configured to sanitize an area around the display device 100. Display device 100 may include a display panel 110 and a controller 130. The display device 100 may optionally include a motion detection device 120. The display device 100 may also include a mechanism for rotating the display panel 110.

Display panel 110 is an electronic display. The electronic display may be a light emitting diode (LED) display, such as a micro LED display. The micro LED display may include sets of LED units. Each set of LED units may include a plurality of visible light emitting LEDs configured to emit visible light in a corresponding visible range (e.g., red, green, and blue light emitting LEDs, or other color combinations of light emitting LEDs), and an ultraviolet light emitting LED configured to emit light in a UV range. The micro LED display may be fabricated by separately growing different types of LEDs (e.g., red, green, blue, and UV light emitting LEDs) on separate wafers and transferring each type of LED from the corresponding wafer to display electronics (e.g., via pick-and-place). Unlike a liquid crystal display (LCD) which can include multiple layers (e.g., one or more plastic polarizer layers, etc.) that may chemically change and become discolored due to UV exposure, a micro LED display can have a simpler structure with a cover (e.g., glass or film having no discoloration issues) covering the LED units. The UV light emitting LEDs may emit UV light in a UV-A (e.g., 315-400 nm), UV-B (e.g., 280-315 nm), and/or UV-C (e.g., 100-280 nm) range. UV light can be used for sanitizing purposes because it can break down chemical bonds and change structure of deoxyribonucleic acid (DNA), ribonucleic acid (RNA), or proteins of microorganisms rendering them no longer infectious. For sanitizing purposes, UV-C light emitting LEDs are typically used. UV-A and UV-B light emitting LEDs can also be used for sanitizing purposes, but may be less effective.

Motion detection device 120 may include a sensor that detects motion. For example, the sensor may be a camera, a photodetector, an infrared sensor, any combination thereof, etc. The motion detection device 120 may also include a transmitter. For example, the motion detection device 120 may include an ultrasonic transmitter to transmit ultrasonic wave and an ultrasonic transducer (e.g., sensor) to receive wave reflections off of objects. The motion detection device 120 may include other types of transmitter and receiver pairs to detect motion.

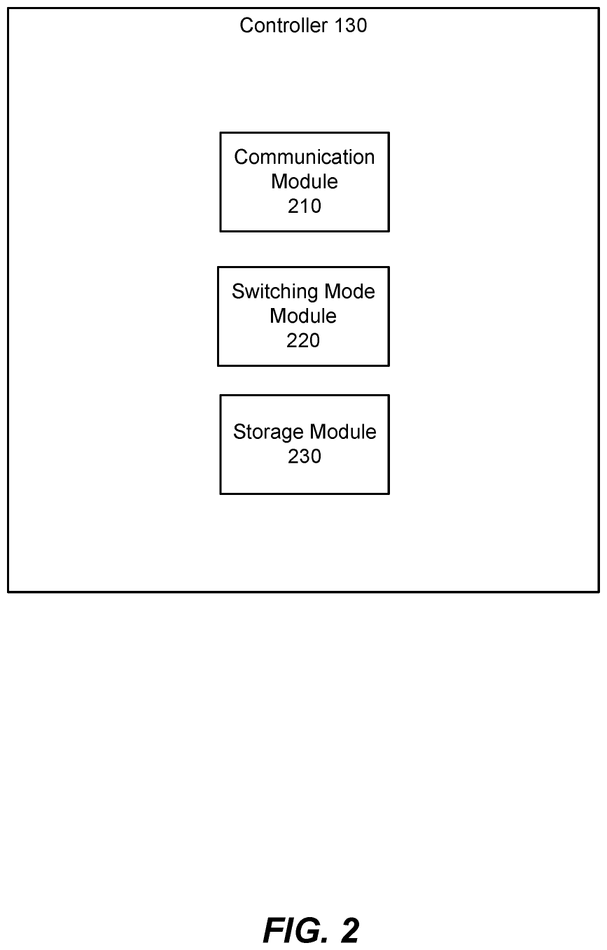

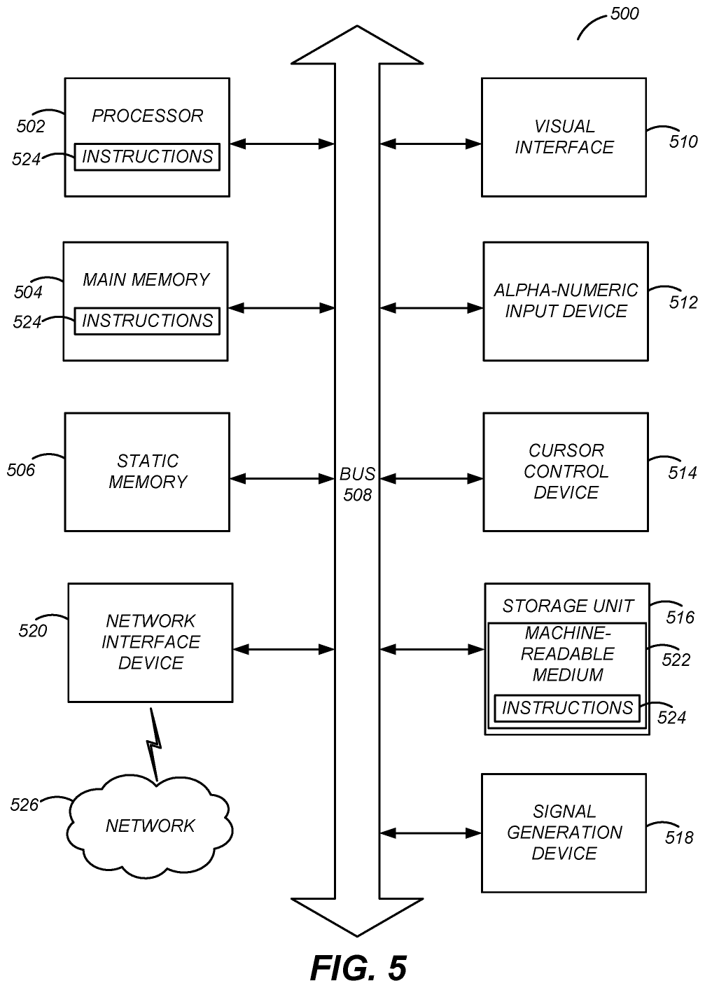

Controller 130 may include a processor and other hardware components (e.g., main memory, static memory, network interface device and/or other components shown in FIG. 5). In some embodiments, controller 130 may be a processor (e.g., the processor as shown in FIG. 5). The controller 130 controls operation of the display device 100. For example, the controller 130 may cause the display panel to operate in a display mode (e.g., display image data with the visible light emitting LEDs) or a cleaning mode (e.g., sanitize a workspace using the UV light emitting LEDs). The controller 130 may communicate with a computing device coupled to the display device 100 and with the display panel 110. For example, the controller 130 may receive image data from the computing device, and the controller 130 may cause the display panel 110 to display image data in a display mode of operation. The controller 130 may determine whether to switch from a display mode to a cleaning mode. For example, the controller 130 may determine that no other image data has been received from the computing device, and retrieve one or more conditions for switching modes of operation to a cleaning mode. An example of a condition for switching modes of operation to a cleaning mode may be that the display device has been idle for a period of time (e.g., display device has not been used to display data) and/or no motion has been detected for a threshold period of time (e.g., indicating that no one is around and it would be safe to switch into a cleaning mode). The controller may determine whether a condition for switching modes of operation to the cleaning mode has been met, and cause the display panel to turn off the plurality of visible light emitting LEDs and turn on each UV light emitting LED in each set of LED units to sanitize an area around the display device in a cleaning mode. The controller 130 may store and retrieve information from storage to assist in determining whether to switch from a display mode to a cleaning mode. The controller 130 will be described in further detail in the detailed description of FIG. 2.

In some embodiments, the controller 130 may instruct the motion detection device 120 to collect sensor data and/or receive sensor data from the motion detection device 120. The sensor data may indicate whether motion was detected in an area surrounding the display device 100. The controller 130 may utilize the sensor data to determine whether or not one or more conditions for switching modes of operation to a cleaning mode of operation has been met. An example of a condition may be that no motion has been detected by a motion detector for a threshold period of time indicating that no one is in the vicinity of the display and it would be safe to switch into a cleaning mode.

In some embodiments, the display device 100 includes a mechanism for rotating the display panel 110. The mechanism may allow the display panel 110 to be rotated in a horizontal direction to scan a larger workspace area. The mechanism may allow the display panel 110 to rotate in a vertical direction to tilt the display panel 110 towards a workspace area that is heavily contacted by users, such as a desk surface, keyboard, mouse, etc. The controller 130 may be configured to instruct the mechanism for rotating the display panel 110 to rotate the display panel 110 in a horizontal and/or vertical direction while the display panel 110 operates in cleaning mode to sanitize a larger area around the display panel. In some embodiments, the controller 130 may be configured to instruct the mechanism for rotating the display panel 110 to rotate the display panel 110 in a horizontal and/or vertical direction while transmitting and/or receiving sensor data from the motion detection device 120 to obtain sensor data in a larger area around the display panel.

In some embodiments, the display device 100 is a large display device used for a conference room setting. The large display device may include a display panel 110 that may be able to sanitize a large portion of a room in a cleaning mode because it is angled and positioned to be seen in almost every angle of the room. The large display device may also include a mechanism for rotating the display panel 110.

FIG. 2 illustrates one embodiment of a controller 130 of a display device 100. The controller 130 includes a communication module 210, a switching mode module 220, and a storage module 230.

The communication module 210 communicates with the display panel 110 and with a computing device. For example, the computing device may include a processor, main memory, static memory, network interface device and/or other components shown in FIG. 5. The communication module 210 may receive a status indicator from the computing device. A status indicator may indicate that the computing device is active, inactive (e.g., sleep mode), or has been turned on (e.g., exiting sleep mode or upon start-up of the computing device). The communication module 210 may receive image data from the computing device. The communication module 210 may transmit instructions to a display panel 110 of the display device 100. The instructions may cause the display panel 110 to operate in a display mode and display the received image data. For example, the instructions may cause the display panel 110 to turn on the visible light emitting LEDs in each set to display the received image data and to turn off each UV light emitting LED in each set. The instructions may cause the display panel 110 to operate in a cleaning mode. For example, the display panel 110 may turn off visible light emitting LEDs in each set and turn on each UV light emitting LED in each set. In some embodiments, the communication module 210 may communicate with the motion detection device 120. For example, the communication module 210 may transmit instructions to or receive sensor data from the motion detection device 120. The instructions may cause the motion detection device 120 to activate transmitters and/or sensors to sense motion and to transmit detected data to the communication module 210. In some embodiments, the transmitters and/or sensors on the motion detection device 120 may always be active and may transmit motion data without the instructions from the communication module 210. The communication module 210 may receive the sensor data from the motion detection device 120. In some embodiments, the communication module 210 may communicate with a mechanism for rotating the display panel 110. For example, the communication module 210 may transmit instructions to the mechanism for rotating the display panel 110. The instructions may instruct the mechanism for rotating the display panel 110 to rotate the display panel 110 in a horizontal and/or vertical direction.

The switching mode module 220 determines whether to operate the display panel 110 in a display mode or a cleaning mode. For example, the display panel 110 may be operated in a display mode based on whether the computing device is turned on, whether the controller 130 is receiving image data from the computing device, or whether a current time of day is within a time range associated with the display mode of operation.

In some embodiments, the switching mode module 220 may determine to operate the display panel in a display mode responsive to an indication that the computing device is turned on. For example, the controller 130 may receive information from the computing device. The switching mode module 220 may identify and determine a value of the status indicator (e.g., what the status indicator represents) based on the received information following a pre-defined convention or protocol to decode information sent by computing device. The switching mode module 220 determines whether the status indicator indicates the computing device has been turned on. In response to determining the status indicator indicates the computing device has been turned on, the switching mode module 220 determines to operate the display panel 110 in a display mode.

In some embodiments, the switching mode module 220 may determine to operate the display panel 110 in a display mode responsive to the controller 130 receiving image data from the computing device. For example, the controller 130 may receive information from the computing device. The switching mode module 220 may identify the information as image data based on the received information following a pre-defined convention or protocol to decode information sent by computing device. The switching mode module 220 may determine to operate the display panel 110 in a display mode as long as image data continues to be sent from the computing device.

The switching mode module 220 may determine that no image data is being sent from the computing device based on determining that the controller 130 has not received any image data from the computing device within a predetermined period of time. The switching mode module 220 may determine to operate the display panel 110 in a display mode responsive to a current time falling within a time range associated with a display mode of operation. For example, the switching mode module 220 may retrieve a current time (e.g., system time from computer system of FIG. 5) and a time range associated with a display mode of operation retrieved from storage (e.g., using storage module 230, main memory and/or storage unit of FIG. 5). The switching mode module 220 may compare the current time (e.g., 10 AM) to the time range to see whether the current time falls within the time range (e.g., 9 AM-5 PM). The switching mode module 220 may determine to operate the panel 110 in a display mode based on determining that the current time is within the time range (e.g., current time 10 AM is within time range of 9 AM-5 PM). In some embodiments, the time range associated with a display mode of operation may be based on user input (e.g., user indicates time range in which they are working). For example, the controller 130 may receive a time range associated with the display mode of operation based on user input and generate a rule based on the time range. The rule may include a time condition based on the time range and an action to cause the display panel to turn off each UV light emitting LED in each set and turn on the visible light emitting LEDs. In some examples, the action may be to turn off each UV light emitting LED in each set without turning on the visible light emitting LEDs (e.g., display panel 110 returns to a display mode and is ready to display image data, but controller 130 has not received image data to be displayed).

In some embodiments, the time range associated with a display mode of operation may be based on tracking when the UV light emitting LEDs are turned on and off. For example, the controller 130 may track times at which each UV light emitting LED in each set is turned on and when each UV light emitting LED in each set is turned off, and determine the time range associated with the display mode of operation based on the times at which each UV light emitting LED in each set is turned on and when each UV light emitting LED in each set is turned off. In some embodiments, the controller 130 may detect over a period of time (e.g., over multiple days, multiple weeks, multiple months, or over another time period) that each UV light emitting LED is turned on and off at approximately the same time. For example, the UV light emitting LED in each set may be turned off at approximately 12 AM -1 AM, turned on after 1 AM, and turned off again at 3 AM until the end of the day (e.g., 12 AM). The controller 130 may determine the time range associated with the display mode as being from 12 AM-1 AM, and 3 AM until the end of the day (e.g., the same range of time at which each UV light emitting LED in each set is turned off, or a range of times outside of a range of time in which each UV light emitting LED in each set is turned on).

As another example, the controller 130 may detect that the UV light emitting LEDs are turned on and off at approximately different times. For example, the controller 130 may detect on one day that the UV light emitting LED in each set may be turned off at approximately 12 AM-1 AM, turned on after 1 AM, and turned off again after 3 AM until the end of that day, and may detect on another day that the UV light emitting LED in each set may be turned off at approximately 12 AM-3 AM, turned on after 3 AM, and turned off again after 5 AM until the end of that day. The controller 130 may determine the time range associated with a display mode as being from 12 AM-1AM and 5 AM until the end of the day (e.g., the same range of time at which each UV light emitting LED in each set is turned off on both days). In some embodiments, the controller 130 may determine the time range associated with a display mode by including an offset. Continuing with the last example, and using a 1 hour offset, the controller may determine a time range associated with a display mode to be from 6 AM until the end of the day.

The switching mode module 220 may determine whether to switch the operation of the display panel 110 from a display mode to a cleaning mode. For example, the display device 100 may be idle for a predetermined period of time (e.g., not receiving image data from a computing device), and the controller 130 may determine whether it is an appropriate time to switch into a cleaning mode. In response to determining that no other image data has been received from the computing device, the switching mode module 220 retrieves one or more conditions for switching modes of operation to a cleaning mode of operation. For example, the display panel 110 may be operated in a cleaning mode based on whether a current time of day is outside a time range associated with a display mode of operation (e.g., as previously described), whether the user scheduled a time or time range for cleaning to take place, or whether no one is present (e.g., no motion detected).

In some embodiments, the switching mode module 220 may determine to operate the display panel 110 in a cleaning mode in response to determining whether a current time of day is outside the time range associated with the display mode of operation. For example, the controller 130 may retrieve a current time of day (e.g., system time from computer system of FIG. 5) and retrieve a time range associated with the display mode of operation from storage (e.g., using storage module 230, main memory and/or storage unit of FIG. 5). The switching mode module 220 may compare the current time of day and the time range, and determine whether the current time of day (e.g., 10 PM) is outside the time range associated with the display mode of operation (e.g., 9 AM-5 PM). In response to determining that the current time of day is outside the time range associated with the display mode of operation (e.g., 10 PM is outside of the time range 9 AM5 PM), determine that the condition of the one or more conditions for switching modes of operation to the cleaning mode of operation has been met.

The switching mode module 220 may determine to operate the display panel 110 in a cleaning mode responsive to the user indicating a time range for a cleaning mode to occur (e.g., user schedules cleaning to occur within a certain time range, or after a start time). As an example, a user can set a time range for cleaning to occur (e.g., time between when users switch in and out of a workspace, or a time after a user leaves a workspace, but before returning to the workspace). The controller 130 may receive a user input indicating a time range when the cleaning mode of operation is to be started and to be ended (e.g., user inputs cleaning should take place between 12 PM-1 PM, or between 10 PM5 AM). The controller 130 may generate a rule based on the user input, the rule including the time range when the cleaning mode of operation is to be started and to be ended (e.g., time range entered by the user, or a determined time range) and an action (e.g., turn on UV light emitting LEDs at a determined intensity). The action may be causing the display panel to turn off the plurality of visible light emitting LEDs in each set and to turn on each UV light emitting LED in each set (e.g., at a determined time range and intensity). The controller 130 may then use the rule (e.g., condition) to determine whether to switch into a cleaning mode of operation. The controller 130 may determine whether a current time of day (e.g., 11 PM) is within the time range of the rule (e.g., 10 PM-5 AM) and based on determining that the current time of day is within the time range of the rule (e.g., 11 PM is between 10 PM-5 AM), causing the display panel to turn off the plurality of visible light emitting LEDs in each set and to turn on each UV light emitting LED in each set (e.g., at a determined time range and intensity).

In some embodiments, the controller 130 may determine a time duration such as when user wants to schedule a time for the cleaning, and may additionally determine an intensity required to satisfy the condition for cleaning. For example, the controller 130 may receive a time duration for the cleaning mode of operation, and determine, based on the time duration, a required intensity for the cleaning mode of operation, and cause the display panel 110 to turn on each UV light emitting LED in each set to the required intensity. In general, a longer duration may require a lower intensity than a shorter duration to sanitize a workspace. As an example, a user may indicate a short time period for cleaning in between users (e.g., 12 PM-1 PM) or the user may indicate a long time period for cleaning when everyone is gone during the day (e.g., 10 PM-5 AM). A longer duration for cleaning may allow for a lower intensity of UV light, so the controller may determine a lower intensity of UV light in response to the user indicating a longer time period (e.g., 10 PM-5 AM) than a shorter time period (e.g., 12 PM-1 PM). The UV light emitting LEDs in the display panel 110 may have been calibrated for an intensity and required duration to sanitize a workspace. For example, the display panel 110 of a display device 100 may be tested after it was manufactured/assembled to determine a list of intensities and corresponding durations to sanitize a given area. For example, the list may indicate that if a duration is X, the intensity of each UV light emitting LED in each set of LED units should be Y to sanitize a given area. The list may be stored using storage module 230 (e.g., using main memory 504 and/or storage unit 516 of FIG. 5). The controller 130 may retrieve the list or intensities and durations using the storage module 230. The switching mode module 220 may determine a required intensity to sanitize a workspace area by identifying a duration that is the same or shorter than the received time duration, and retrieving the corresponding intensity as the required intensity. For instance, in response to a user indicating that cleaning can take place between 1 AM-3 AM, the time duration for cleaning may be 2 hours. The controller 130 may retrieve the list and may identify a duration on the list that is either the time duration (e.g., 2 hours) or one that is shorter than the time duration (e.g., 100 minutes, or some other time duration less than 2 hours). The controller 130 retrieves the corresponding intensity associated with the identified time duration (e.g., 2 hours, 100 minutes, etc.). The controller 130 may determine the time duration and the intensity to drive each UV light emitting LED of each set of LED units to be an identified time duration (e.g., 2 hours, 100 minutes, etc.) and corresponding intensity that is an entry on the list.

For safety reasons, the cleaning mode should not be operated while a user is in view of the display panel 110 as UV light may damage human skin and eyes. Before operating the display panel 110 in a cleaning mode, it would be advantageous to check whether there is anyone in the room. One way for doing so is to utilize the motion detection device 120. Also, if the display device 100 includes a rotating mechanism, a larger area can be scanned for the presence of motion to provide additional safety (e.g., no motion indicating that no one is present).

Before operating the display panel 110 in a cleaning mode, the switching mode module 220 may check whether a motion detection device 120 has detected any motion (e.g., if no motion is detected it is likely that no one is present and it may be safe to operate in cleaning mode). In one embodiment, the switching mode module 220 may determine to operate the display panel 110 in a cleaning mode responsive to no motion being detected within a threshold period of time. The threshold period of time may be a predetermined period of time (e.g., preset default time, user specified amount of time, etc.). The controller 130 may receive data from a motion detection device 120. The switching mode module 220 may analyze the data from the motion detection device 120 within the threshold period of time (e.g., 1 minute, 5 minutes, or any other amount of time) to determine whether there is motion detected. For example, if the motion detection device 120 is a camera and the threshold period of time is 5 minutes, the switching mode module 220 may analyze captured image data from the camera within the last 5 minutes to identify objects in the images and determine whether any objects in the images are moving. The switching mode module 220 may determine whether an object is moving based on whether the object changes location with respect to time (e.g., from one image frame to another image frame). The switching mode module 220 may determine that no motion has been detected based on there being no change in location of objects from one image frame to another image frame within the threshold period of time (e.g., last 5 minutes), or based on the change in location being within a threshold amount (e.g., within some error range, not enough to indicate motion of an object). The switching mode module 220 may determine to operate the display panel 110 in a cleaning mode in response to determining, using the motion detection device 120, that no motion has been detected within a threshold period of time before and including a current time of day. In some embodiments, the switching mode module 220 may use a combination of conditions to determine whether to operate the display panel 110 in a cleaning mode. For example, after determining whether a current time of day is outside the time range associated with the display mode of operation, the motion detection module 220 may determine that no motion has been detected within a threshold period of time before and including a current time of day before starting a clean mode for the display panel 110.

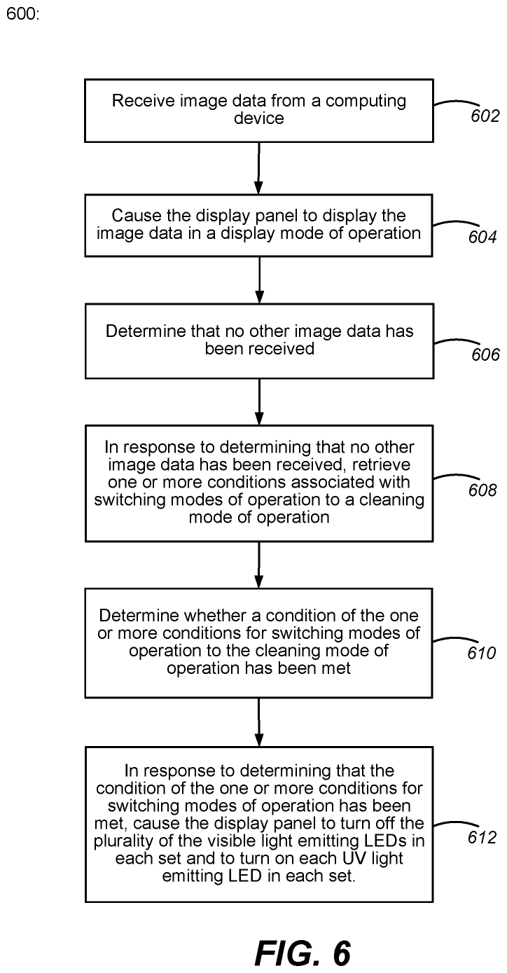

The storage module 230 stores information for the controller 130 of the display device 100. For example, the storage module 230 may use a data structure illustrated in FIG. 6 to store the conditions and actions. The controller 130 may store a time range associated with the display mode of operation using the storage module 230 as a condition associated with an action to cause the display panel 110 to display image data. The controller 130 may retrieve the time range associated with the display mode of operation using the storage module 230 to use when determining whether conditions are satisfied to perform the associated action to cause the display panel 110 to display image data. The storage module 230 may store the list of intensities and durations of UV light emitting LEDs required to sanitize a workspace. The controller 130 may retrieve the stored list of intensities and durations to use when determining what intensity to turn on each UV light emitting LEDs of each set for a given duration. For example, the controller 130 may retrieve the list (e.g., intensities and durations required to sanitize an area) using the storage module 230. The controller 130 may determine a duration and an intensity to sanitize a workspace area that fit in a scheduled cleaning input by a user. For example, in response to the user indicating that a cleaning can occur during a 2 hour time frame, the controller 130 may retrieve the list and may identify a duration on the list that is either the time duration (e.g., 2 hours) or one that is shorter than the time duration (e.g., 100 minutes, or some other time duration less than 2 hours). The controller 130 may retrieve the corresponding intensity from the list that is associated with the identified time duration. The controller 130 may determine the time duration and the intensity to drive each UV light emitting LED of each set of LED units to be the identified time duration and corresponding intensity that is an entry on the list.

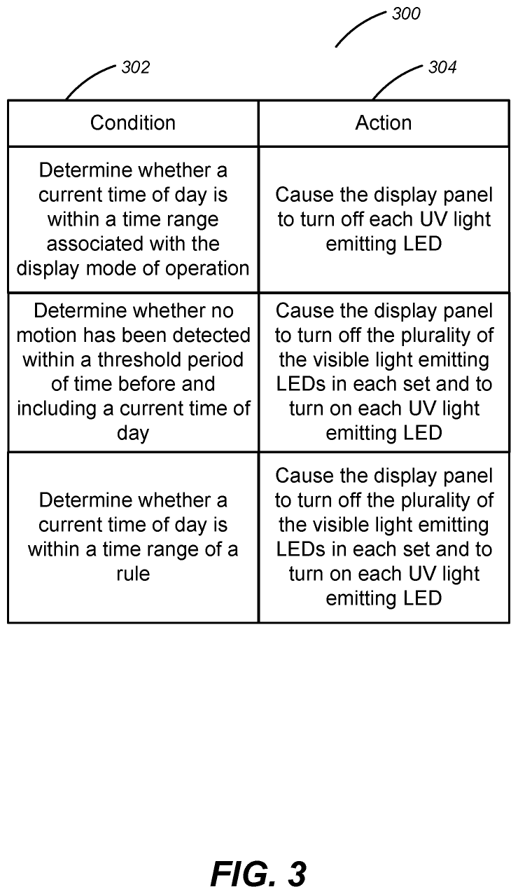

FIG. 3 illustrates a data structure 300 and example entries for a condition for switching mode of operations for a display panel 110, in accordance with some embodiments of this disclosure. The data structure 300 includes a condition 302 and an action 304. For example, a first condition may be “determine whether a current time of day is within a time range associated with the display mode of operation” and the corresponding action may be “cause the display panel to turn off each UV light emitting LED.” A second condition may be “determine whether no motion has been detected within a threshold period of time before and including a current time of day” and the corresponding action may be “cause the display panel to turn off the plurality of the visible light emitting LEDs in each set and to turn on each UV light emitting LED.” A third condition may be “determine whether a current time of day is within a time range of a rule” and the corresponding action may be “cause the display panel to turn off the plurality of the visible light emitting LEDs in each set and to turn on each UV light emitting LED.” The data structure may be used to store example entries using the storage module 230.

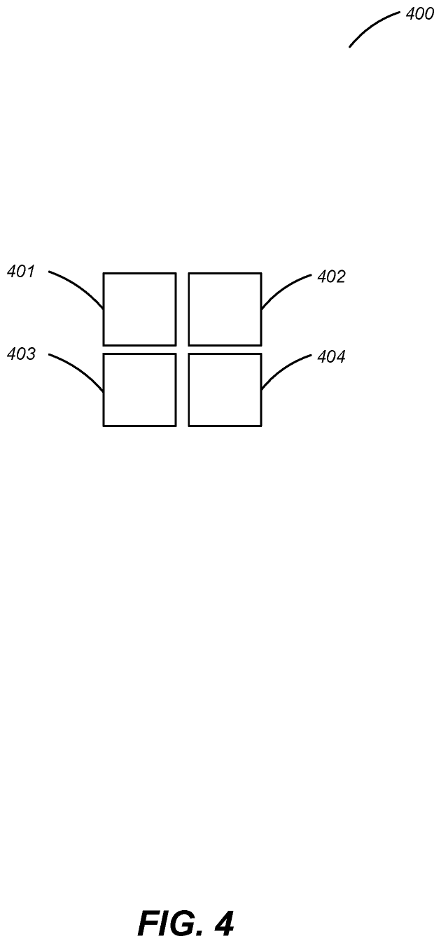

FIG. 4 illustrates one embodiment of a set 400 of LED units of a display panel 110 of a display device 100. The set of LED units includes four LED units: LED unit 401, LED unit 402, LED unit 403, and LED unit 404. The set 400 of LED units includes three visible light emitting LED units and one UV light emitting LED unit. For example, the set 400 of LED units may include three visible light emitting LEDs and a UV light emitting LED. The LED unit 401 may be a blue light emitting LED, LED unit 402 may be a green light emitting LED, LED unit 403 may be a UV light emitting LED, and LED unit 404 may be a red light emitting LED. The LED units in the set of LED units are divided into two rows of two units, the rows positioned on top of each other. For example, a first row of LED units are LED unit 401 and LED unit 402. A second row of LED units are LED unit 403 and LED unit 404. The first row of LED units (e.g., LED unit 401 and LED unit 402) are positioned on top of the second row of LED units (e.g., LED unit 403 and LED unit 404).

In some embodiments, the UV light emitting LED unit (e.g., LED unit 403) may be configured to alternate between emitting UV light and white light, and the controller 130 may be configured to operate the UV light emitting unit to switch between emitting UV light and white light. For example, the controller 130 may cause the display panel 110 to operate in a display mode (e.g., display image data with the visible light emitting LEDs) and to operate the UV light emitting unit to emit white light for additional brightness in the display mode. The controller 130 may be configured to cause the display panel 110 to operate in a cleaning mode (e.g., sanitize a workspace with the UV light emitting LEDs) and to operate the UV light emitting unit to emit UV light in the cleaning mode.

### COMPUTING MACHINE ARCHITECTURE

The actions of FIG. 5 may be implemented using the components of a device illustrated in FIG. 2. FIG. 5 is a block diagram illustrating components of an example machine able to read instructions from a machine-readable medium and execute them in a processor (or controller). Specifically, FIG. 5 shows a diagrammatic representation of a machine in the example form of a computer system 500 within which program code (e.g., software) for causing the machine to perform any one or more of the methodologies discussed herein may be executed. The program code may be comprised of instructions 524 executable by one or more processors 502. In alternative embodiments, the machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The machine may be a server computer, a client computer, a personal computer (PC), a tablet PC, a set-top box (STB), a personal digital assistant (PDA), a cellular telephone, a smartphone, a web appliance, a network router, switch or bridge, or any machine capable of executing instructions 524 (sequential or otherwise) that specify actions to be taken by that machine. Further, while only a single machine is illustrated, the term “machine” shall also be taken to include any collection of machines that individually or jointly execute instructions 524 to perform any one or more of the methodologies discussed herein.

The example computer system 500 includes a processor 502 (e.g., a central processing unit (CPU), a graphics processing unit (GPU), a digital signal processor (DSP), one or more application specific integrated circuits (ASICs), one or more radio-frequency integrated circuits (RFICs), or any combination of these), a main memory 504, and a static memory 506, which are configured to communicate with each other via a bus 508. The computer system 500 may further include visual display interface 510. The visual interface may include a software driver that enables displaying user interfaces on a screen (or display). The visual interface may display user interfaces directly (e.g., on the screen) or indirectly on a surface, window, or the like (e.g., via a visual projection unit). For ease of discussion the visual interface may be described as a screen. The visual interface 510 may include or may interface with a touch enabled screen. The computer system 500 may also include alphanumeric input device 512 (e.g., a keyboard or touch screen keyboard), a cursor control device 514 (e.g., a mouse, a trackball, a joystick, a motion sensor, or other pointing instrument), a storage unit 516, a signal generation device 518 (e.g., a speaker), and a network interface device 520, which also are configured to communicate via the bus 508.

The storage unit 516 includes a machine-readable medium 522 on which is stored instructions 524 (e.g., software) embodying any one or more of the methodologies or functions described herein. The instructions 524 (e.g., software) may also reside, completely or at least partially, within the main memory 504 or within the processor 502 (e.g., within a processor's cache memory) during execution thereof by the computer system 500, the main memory 504 and the processor 502 also constituting machine-readable media. The instructions 524 (e.g., software) may be transmitted or received over a network 526 via the network interface device 520.

While machine-readable medium 522 is shown in an example embodiment to be a single medium, the term “machine-readable medium” should be taken to include a single medium or multiple media (e.g., a centralized or distributed database, or associated caches and servers) able to store instructions (e.g., instructions 524). The term “machine-readable medium” shall also be taken to include any medium that is capable of storing instructions (e.g., instructions 524) for execution by the machine and that cause the machine to perform any one or more of the methodologies disclosed herein. The term “machine-readable medium” includes, but not be limited to, data repositories in the form of solid-state memories, optical media, and magnetic media. The components of FIG. 5 may be used to implement the actions of FIGS. 3 and 6.

FIG. 6 illustrates one example of actions for a controller 130 of a display device 100 to switch operation of a display panel 110 from a display mode of operation to a cleaning mode of operation, in accordance with some embodiments of this disclosure. At 602, a controller receives image data from a computing device. As illustrated in FIG. 5, a computing device may include a network interface device (e.g., network interface device 526). The display device 100 may also have a built-in computing device (e.g., controller 130) with at least some of the components illustrated in FIG. 5. Both the computing device and the display device 110 may include a network interface device (e.g., network interface device 520). The network interface device may enable wired and/or wireless communications to establish a connection between the computing device and the display device 110.

At 604, the controller 130 causes the display panel 110 to display the image data in a display mode of operation. For example, the controller 130 may cause the display panel 110 to turn on the visible light emitting LEDs in each set to display the image data, and to turn off each UV light emitting LED of each set.

At 606, the controller 130 determines that no other image data has been received. For example, the user may have left for the day and the computing device may be turned off or in a sleep mode.

At 608, the controller 130, in response to determining that no other image data has been received, retrieves one or more conditions associated with switching modes of operation to a cleaning mode of operation. The controller 130 may retrieve the one or more conditions using the storage module 230 (e.g., using main memory 504 and/or storage unit 516 of FIG. 5).

At 610, the controller 130 determines whether a condition of the one or more conditions for switching modes of operation to the cleaning mode of operation has been met. For example, the display panel 110 may be operated in a cleaning mode based on whether a current time of day is outside a time range associated with a display mode of operation, whether the user scheduled a time or time range for cleaning to take place, or whether no motion is detected.

At 612, the controller 130, in response to determining that the condition of the one or more conditions for switching modes of operation has been met, causes the display panel to turn off the plurality of the visible light emitting LEDs in each set and to turn on each UV light emitting LED in each set. The controller 130 may also receive a time duration and determine based on the time duration an intensity to turn on each UV light emitting LED in each set.

### ADDITIONAL CONFIGURATION CONSIDERATIONS

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of components, modules, or mechanisms. Modules may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. As used herein, “hardware-implemented module” refers to a hardware module. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs).)

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. It should be understood that these terms are not intended as synonyms for each other. For example, some embodiments may be described using the term “connected” to indicate that two or more elements are in direct physical or electrical contact with each other. In another example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for sanitizing a workspace using a display device through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

1.-20. (canceled)

21. A display device comprising:
a display panel comprising a plurality of sets of light emitting diode (LED) units, wherein each set of LED units from the plurality of sets of LED units includes a plurality of visible light emitting LEDs configured to emit visible light in a corresponding visible range, and an ultraviolet (UV) light emitting LED configured to emit light in a UV range; and
a controller configured to:
operate the display panel in a display mode by turning on the plurality of visible light emitting LEDs included in the plurality of sets of LED units to display image data and turning off the UV light emitting LED included in the plurality of LED units; and
switch operation from the display mode of the display panel to a cleaning mode of the display panel by turning off the plurality of visible light emitting LEDs included in the plurality of sets of LED units and turning on the UV light emitting LED included in the plurality of LED units.

22. The display device of claim 21, wherein the controller is further configured to:
receive the image data from a computing device, the image data displayed during the display mode;
determine that no other image data is received; and
retrieve one or more conditions for switching modes of operation from the display mode to the cleaning mode responsive to determining that no other image data is received;
wherein the controller switches the display panel from the display mode to the cleaning mode responsive to determining that the one or more conditions for switching modes of operation from the display mode to the cleaning mode is met.

23. The display device of claim 21, wherein the controller is further configured to:
cause the display panel to turn off each UV light emitting LED in each of the plurality of sets of LED units responsive to receiving an indication that a computing device that is connected to the display device is turned on.

24. The display device of claim 21, wherein the controller is further configured to:
determine whether a current time of day is within a time range associated with the display mode; and
cause the display panel to turn off each UV light emitting LED in each of the plurality of sets of LED units responsive to determining that the current time of day is within the time range.

25. The display device of claim 24, wherein the controller is further configured to:
receive the time range associated with the display mode based on user input; and
generate a rule based on the received time range, the rule including a time condition based on the received time range and an action to cause the display panel to turn off each UV light emitting LED in each of the plurality of sets of LED units.

26. The display device of claim 24, wherein the controller is further configured to:
track times at which each UV light emitting LED in each of the plurality of sets of LED units is turned on and when each UV light emitting LED in each of the plurality of sets of LED units is turned off; and
determine the time range based on the tracked times.

27. The display device of claim 22, further comprising:
retrieve a current time of day and a time range associated with the display mode;
determine whether the current time of day is outside the time range associated with the display mode; and
determine that the one or more conditions for switching modes of operation from the display mode to the cleaning mode is met responsive to determining that the current time of day is outside the time range associated with the display mode.

28. The display device of claim 22, wherein the controller is further configured to:
receive a user input indicating a time range when the cleaning mode starts and ends;
generate a rule based on the user input, the rule including the time range when the cleaning mode starts and ends and an action, the action causing the display panel to turn off the plurality of the visible light emitting LEDs in each of the plurality of sets of LED units and to turn on each UV light emitting LED in each of the plurality of sets of LED units;
determine whether a current time of day is within the time range of the rule; and
cause the display panel to turn off the plurality of the visible light emitting LEDs in each of the plurality of sets of LED units and to turn on each UV light emitting LED in each of the plurality of sets of LED units responsive to determining that the current time of day is within the time range of the rule.

29. The display device of claim 22, further comprising a motion detection device and the controller further configured to:
determine that no motion has been detected within a threshold period of time which satisfies the one or more conditions for switching modes of operation from the display mode to the cleaning mode.

30. The display device of claim 21, wherein the controller is further configured to:
receive a time duration for the cleaning mode of operation;
determine a required intensity for the cleaning mode based on the time duration; and
cause the display panel to turn on each UV light emitting LED in each of the plurality of sets of LED units to the required intensity.

31. The display device of claim 21, wherein each of the plurality of sets of LED units includes four LED units including a red light emitting LED, a green light emitting LED, a blue light emitting LED, and the UV light emitting LED, and wherein the LED units in each set of LED units are divided into two rows of two units, the rows positioned on top of each other.

32. A method for switching modes of operation for a display panel comprising a plurality of sets of light emitting diode (LED) units, wherein each set of LED units comprises a plurality of visible light emitting LEDs configured to emit visible light in a corresponding visible range, and an ultraviolet (UV) light emitting LED configured to emit light in a UV range, the method comprising:
operating the display panel in a display mode by turning on the plurality of visible light emitting LEDs included in the plurality of sets of LED units to display image data and turning off the UV light emitting LED included in the plurality of LED units; and
switching operation from the display mode of the display panel to a cleaning mode of the display panel by turning off the plurality of visible light emitting LEDs included in the plurality of sets of LED units and turning on the UV light emitting LED included in the plurality of LED units.

33. The method of claim 32, further comprising:
receiving the image data from a computing device, the image data displayed during the display mode;
determining that no other image data is received;
retrieving one or more conditions for switching modes of operation from the display mode to the cleaning mode responsive to determining that no other image data is received,
wherein the operation from the display mode to the cleaning mode is switched responsive to determining that the one or more conditions for switching modes of operation from the display mode to the cleaning mode is met.

34. The method of claim 32, further comprising:
causing the display panel to turn off each UV light emitting LED in each of the plurality of sets of LED units responsive to receiving an indication that a computing device that is connected to the display device is turned on.

35. The method of claim 32, further comprising:
determining whether a current time of day is within a time range associated with the display mode; and
causing the display panel to turn off each UV light emitting LED in each of the plurality of sets of LED units responsive to determining that the current time of day is within the time range.

36. The method of claim 35, further comprising:
receiving the time range associated with the display mode based on user input; and
generating a rule based on the received time range, the rule including a time condition based on the received time range and an action to cause the display panel to turn off each UV light emitting LED in each of the plurality of sets of LED units.

37. The method of claim 35, further comprising:
tracking times at which each UV light emitting LED in each of the plurality of sets of LED units is turned on and when each UV light emitting LED in each of the plurality of sets of LED units is turned off; and
determining the time range based on the tracked times.

38. The method of claim 33, further comprising:
retrieving a current time of day and a time range associated with the display mode;
determining whether the current time of day is outside the time range associated with the display mode; and
determining that the one or more conditions for switching modes of operation from the display mode to the cleaning mode is met responsive to determining that the current time of day is outside the time range associated with the display mode.

39. The method of claim 33, further comprising:
receiving a user input indicating a time range when the cleaning mode starts and ends;
generating a rule based on the user input, the rule including the time range when the cleaning mode starts and ends and an action, the action causing the display panel to turn off the plurality of the visible light emitting LEDs in each of the plurality of sets of LED units and to turn on each UV light emitting LED in each of the plurality of sets of LED units;
determining whether a current time of day is within the time range of the rule; and
causing the display panel to turn off the plurality of the visible light emitting LEDs in each of the plurality of sets of LED units and to turn on each UV light emitting LED in each of the plurality of sets of LED units responsive to determining that the current time of day is within the time range of the rule.

40. A non-transitory computer-readable medium comprising memory with instructions encoded thereon for switching modes of operation for a display panel comprising a plurality of sets of light emitting diode (LED) units, wherein each set of LED units comprises a plurality of visible light emitting LEDs configured to emit visible light in a corresponding visible range, and an ultraviolet (UV) light emitting LED configured to emit light in a UV range, the instructions when executed by the one or more processors cause the one or more processors to perform operations comprising:
operating the display panel in a display mode by turning on the plurality of visible light emitting LEDs included in the plurality of sets of LED units to display image data and turning off the UV light emitting LED included in the plurality of LED units; and
switching operation from the display mode of the display panel to a cleaning mode of the display panel by turning off the plurality of visible light emitting LEDs included in the plurality of sets of LED units and turning on the UV light emitting LED included in the plurality of LED units.
